(19) United States
(12) Patent Application Publication
(10) Pub. No.: US 2023/0046673 A1
(43) Pub. Date: Feb. 16, 2023

(54) TREATMENT OF OBSTRUCTIVE SLEEP APNEA

(71) Applicant: MIRAKI INNOVATION THINK TANK LLC, Cambridge, MA (US)

(72) Inventors: Christopher VELIS, Lexington, MA (US); Karen E. MILLER, South Dartmouth, MA (US); Emilia JAVORSKY, Watertown, MA (US); Rainuka GUPTA, Newton, MA (US); Tarik CHAUDHRY, Boston, MA (US)

(73) Assignee: MIRAKI INNOVATION THINK TANK LLC, Cambridge, MA (US)

(21) Appl. No.: 17/785,730

(22) PCT Filed: Dec. 21, 2020
(86) PCT No.: PCT/US20/66355

§ 371 Date: Jun. 15, 2022

Related U.S. Application Data
(60) Provisional application No. 62/953,683, filed on Dec. 26, 2019.

(51) Int. Cl. A61F 7/12; A61F 5/56; A61K 9/08

(52) U.S. Cl. CPC A61F 7/123; A61F 5/56; A61K 9/08; A61F 2007/126; A61B 2090/0463

(57) ABSTRACT

The present invention provides a cold solution and method for treating obstructive sleep apnea. The cold solution can comprise liquid and/or solid ice particles. In one embodiment, the method comprises delivering a cold solution to target tissue where the target tissue can be fat tissue located in one or more of an airway, a neck, a thorax, a diaphragm, a face, or an abdomen.

### TECHNICAL FIELD

The invention relates to the treatment of obstructive sleep apnea using a cold solution.

### BACKGROUND

Obstructive sleep apnea is a potentially serious sleep disorder that causes breathing to repeatedly stop and start during sleep. There are several types of sleep apnea, but the most common is obstructive sleep apnea, and this type of apnea occurs when the throat muscles intermittently relax and the fat tissue in the airway or tongue block the airway during sleep.

Risk factors often include, among others, excess weight. Most but not all people with obstructive sleep apnea are overweight. Fat deposits around the upper airway may obstruct breathing. Additionally, excess fat around the circumference of the neck and abdomen may also impact breathing.

There are several treatments available for OSA including a device that uses positive pressure to keep the airway open during sleep, however these devices can be cumbersome resulting in inconsistent or nonuse. Another treatment is a mouthpiece device to maintain the lower jaw in a forward position during sleep, again with inconsistent or nonuse. Also, surgery may be an option, but this is invasive and typically a last resort. Accordingly, improved devices and methods for treating obstructive sleep apnea are needed.

### SUMMARY

The present invention provides a cold solution and method for treating obstructive sleep apnea. The cold solution can be substantially liquid, substantially solid, or a slurry comprising both liquid and solid ice particles. In some embodiments, the method comprises delivering a cold solution to target tissue where the target tissue comprises fat tissue in one or more of an airway, a neck, a thorax, a diaphragm, a face, or an abdomen. The method can include delivering the cold solution to one or more sites in the target tissue. The method can further comprise cooling the target tissue via the cold solution. The method can further comprise performing at least one of pre-, peri- or post-procedure imaging and/or measurements. The method can further comprise creating a treatment plan. The method can further comprise removing at least a portion of the cold solution from the target tissue, for example where the removing comprises withdrawing the cold solution via a syringe, withdrawing the cold solution via suction, and/or absorbing the cold solution.

In some embodiments, delivering a cold solution can comprise injecting a cold solution to the target tissue such as one or more sites in the target tissue. The injecting can be performed via a delivery device. The delivery device can comprise a cannula. The delivery device can comprise a syringe having a single needle. The syringe can comprise an array of needles. The array of needles can be expandable. The delivery device can comprise a balloon. The balloon can be configured to contain the cold solution prior to delivery. The balloon can be configured to protect non-target tissue from exposure to the cold solution. The injecting can comprise injecting one or more boluses of cold solution. The injecting can comprise injecting a cold solution in a pattern.

In some embodiments, the delivering a cold solution can comprise circulating a cold solution proximate to the target tissue. In some embodiments, the delivering a cold solution can comprise topically contacting a cold solution to the target tissue.

In some embodiments, the target tissue comprises the airway. The airway comprises an anterolateral upper airway; pharyngeal fat pads; parapharyngeal fat pads; fat located within the tongue; and a soft palate, and the delivering a cold solution to target tissue can comprise delivering a cold solution to one or more portions of the airway.

In some embodiments, the target tissue comprises the neck. The neck comprises subcutaneous fat in an outer circumference of the neck, and the delivering a cold solution to target tissue can comprise delivering a cold solution to subcutaneous fat in at least a portion of the outer circumference of the neck, to one or more portions of the outer circumference of the neck, or to the entire outer circumference of the neck. The neck comprises brown fat, and the delivering a cold solution to target tissue can comprise delivering a cold solution to at least a portion of the brown fat.

In some embodiments, the target tissue comprises the abdomen. The abdomen comprises subcutaneous fat, and the delivering a cold solution to target tissue can comprise delivering a cold solution to at least a portion of the subcutaneous fat. The abdomen comprises brown fat, and the delivering a cold solution to target tissue can comprise delivering a cold solution to at least a portion of the brown fat. The abdomen comprises visceral fat, and the delivering a cold solution to target tissue can comprise delivering a cold solution to at least a portion of the visceral fat.

In some embodiments, the target tissue comprises the thorax. The thorax comprises epicardial fat, and the delivering a cold solution to target tissue can comprise delivering a cold solution to at least a portion of the epicardial fat.

In some embodiments, the target tissue comprises the diaphragm. The diaphragm comprises ectopic fat, and the delivering a cold solution to target tissue can comprise delivering a cold solution to at least a portion of the ectopic fat.

In some embodiments, the target tissue comprises the face. The face comprises subcutaneous fat, and the delivering a cold solution to target tissue can comprise delivering a cold solution to at least a portion of the subcutaneous fat.

In some embodiments, the method further comprises toning a muscle in the target tissue. In some embodiments, the method further comprises providing a positive airway pressure device. In some embodiments, the method further comprises providing a mouthguard. In some embodiments, the method further comprises providing an airway stimulator.

The method can further treat one or more of asthma, diabetes, atrial fibrillation, stroke, and nonalcoholic steatohepatitis.

In some embodiments, a method of treating obstructive sleep apnea comprises delivering a cold solution to target tissue wherein the target tissue comprises one or more of airway fat, subcutaneous fat, visceral fat, brown fat, epicardial fat, or ectopic fat.

In some embodiments, the cold solution comprises water. In some embodiments, the cold solution comprises water and one or more additives. The cold solution can comprise one or more of a salt, a sugar or a thickener. The cold solution can comprise an osmolality of less than about 2,200 milli-Osmoles/kilogram. The cold solution can comprise an osmolality of less than about 1,000 milli-Osmoles/kilogram. The cold solution can comprise an osmolality of less than about 600 milli-Osmoles/kilogram. The cold solution can comprise a pH of about 4.5 to about 9.

In some embodiments, the cold solution comprises no ice. In some embodiments, the cold solution comprises ice particles at a concentration of about 2% to about 70%. In some embodiments, the cold solution comprises ice particles at a concentration of about 20% to about 50%. In some embodiments, the cold solution comprises ice particles at a concentration of about 71% to about 100%. In some embodiments, the cold solution comprises ice particles at a concentration of at least about 95%. In some embodiments, the cold solution comprises substantially ice.

### BRIEF DESCRIPTION OF THE DRAWINGS

FIG. 1A is a diagram of subcutaneous fat locations in the body.

FIG. 1B is a diagram of subcutaneous and visceral fat locations within the abdominal area.

FIG. 1C is a diagram of brown fat tissue locations in the body.

FIG. 2 is a view of an example device for delivering a cold solution to fat tissue.

FIG. 3 is a diagram a cold solution being delivered to subcutaneous fat tissue.

FIG. 4 is a view of an example device for delivering a cold solution to fat tissue.

### DETAILED DESCRIPTION

The present invention provides a cold solution and method for treating obstructive sleep apnea. The cold solution can include a liquid and/or solid ice particles. For example, the cold solution can be substantially liquid, substantially solid, or a slurry, comprising both liquid and solid ice particles. The method includes delivering a cold solution to target tissue where the target tissue can be lipid rich tissue such as adipose or fat tissue. Target tissue can include fat tissue located in one or more of an airway, a neck, a thorax, a diaphragm, a face, or an abdomen. By providing a cold solution to the fat tissue, it effectively crystallizes the fat cells and induces apoptosis, a natural cell death which, in turn, reduces the fat tissue. Additionally, the reduction of fat tissue may also cause a collagen response thereby tightening the skin proximate the target tissue. In brown fat, a cold solution can cause non-shivering thermogenesis in the tissue thereby causing the body to burn calories.

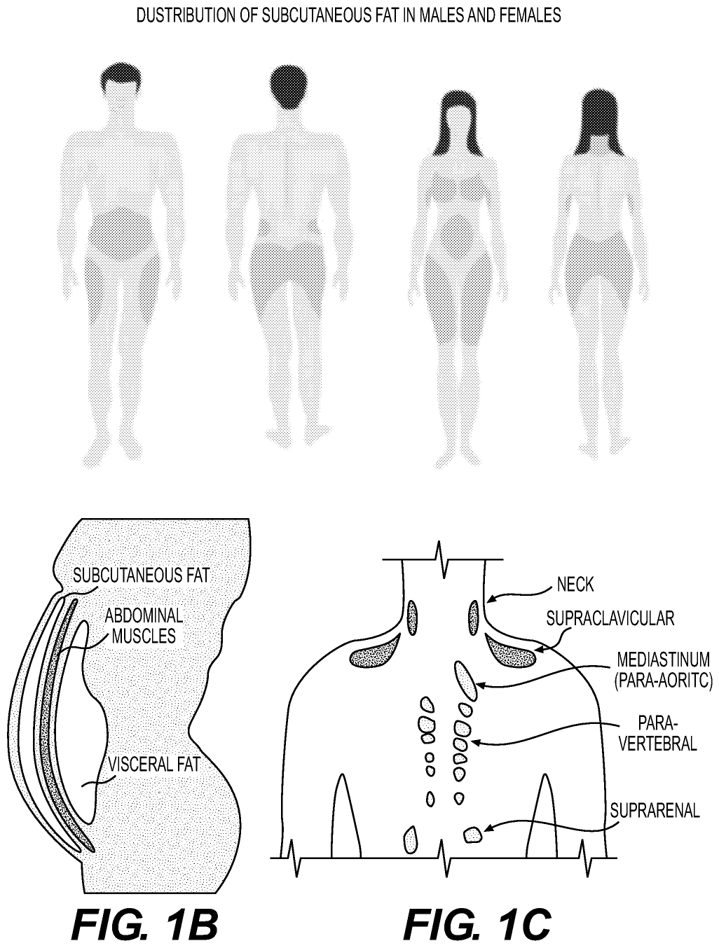

Various locations in the body include various types of fat, including, subcutaneous fat, including superficial and deep layers and sublayers and compartments therein (as shown in FIGS. 1A and 1B), visceral fat (as shown in FIG. 1B), brown fat (as shown in FIG. 1C), epicardial fat, ectopic fat, and airway fat. Subcutaneous fat can be under the skin and above muscle located for example, in the face (such as in the cheeks), neck, submental area under chin, jowls, eyelids, posterior neck (buffalo hump), back, shoulders, arms, triceps, biceps, forearms, hands, chest, breasts, abdomen, abdominal etching and sculpting, flanks (love handles), lower back, buttocks (banana roll), hips (saddle bags), anterior and posterior thighs, inner thighs, mons pubis, vulva, knees, calves, shin, pretibial area, ankles and feet. Visceral fat can be located under the abdominal muscle and may surround internal organs. Brown fat can be can located in the neck, proximate to the clavicle bones (supraclavicular), proximate to the heart (mediastinum), along the spinal cord (paravertebral) and proximate to the kidneys (suprarenal). Intrathoracic fat or epicardial fat can be located in the thorax. Ectopic fat can be located in muscle tissue such as muscle tissue in the diaphragm. Airway fat can be located in any of the airway locations including anterolateral upper airway, pharyngeal fat pads (for example, fatty deposits in the laryngopharynx, nasopharynx, oropharynx, and palatopharynx), parapharyngeal fat pads (for example, fatty deposits in the retropalatal and retroglossal regions), fat located within the tongue (e.g., within the posterior tongue), and soft palate. The target tissue can include one or more types of fat in one or more body areas and any combination thereof.

In some embodiments, the cold solution may include water. In some embodiments, the cold solution may include water and one or more additives. In some embodiments, the one or more additives are inactive, biocompatible ingredients, including any substance (at or below their respective concentrations) in the FDA GRAS list, which is incorporated by reference in its entirety herein. In some embodiments, the additives comprise one or more of a salt, a sugar, and a thickener.

In some aspects, the cold solution comprises potassium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass. In some aspects, the cold solution comprises calcium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass. In some aspects, the cold solution comprises sodium chloride at about 2.25% by mass or lower, for example at about 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1, 0.05 or 0% by mass. In some aspects, the cold solution comprises magnesium chloride at about 0.02% by mass or lower, for example, 0.19, 0.18, 0.17, 0.16, 0.15, 0.14, 0.13, 0.12, 0.11, 0.10, 0.09, 0.08, 0.07, 0.06, 0.05, 0.04, 0.03, 0.02, 0.01, or 0% by mass.

In some aspects, the cold solution comprises sucrose at about 5% by mass or lower, for example at about 5, 4.5, 4, 3.5, 3, 2.5, 2, 1.5, 1, 0.5, or 0% by mass. In some aspects, the cold solution comprises dextrose at about 5.6% by mass or lower, for example at about 5.5, 5, 4.5, 4, 3.5, 3, 2.5, 2, 1.5, 1, 0.5, or 0% by mass. In some aspects, the cold solution comprises mannitol at about 4.95% by mass or lower, for example at about 4.5, 4, 3.5, 3, 2.5, 2, 1.5, 1, 0.5, or 0% by mass. In some aspects, the cold solution comprises lactose at about 0.45% by mass or lower, for example at about 0.4, 0.35, 0.3, 0.25, 0.2, 0.15, 0.1, 0.05, or 0% by mass. In some aspects, the cold solution comprises sorbitol at about 4.7% by mass or lower, for example at about 4.5, 4, 3.5, 3, 2.5, 2, 1.5, 1, 0.5, or 0% by mass. In some aspects, the cold solution comprises glycerol at about 2% by mass or lower, for example at about 1.9, 1.8, 1,7, 1.6, 1.5, 1.4, 1.3, 1.2, 1.1, 1.0, 0.9, 0.8, 0.7, 0.6, 0.5, 0.4, 0.3, 0.2, 0.1, 0.05 or 0% by mass.

In some aspects, the cold solution comprises hetastarch at about 6% by mass or lower, for example at about 5.5, 5, 4.5, 4, 3.5, 3, 2.5, 2, 1.5, 1, 0.5, or 0% by mass. In some aspects, the cold solution comprises pectin at about 16.7% by mass or lower, for example at about 16, 15, 10, 9, 8, 7, 6, 5, 4, 3, 2, 1 or 0% by mass. In some aspects, the cold solution comprises polyethylene glycol at about 20% by mass or lower, for example at about 20, 15, 10, 9, 8, 7, 6, 5, 4, 3, 2, 1 or 0% by mass. In some aspects, the cold solution comprises gelatin at about 16% by mass or lower, for example at about 15, 10, 9, 8, 7, 6, 5, 4, 3, 2, 1 or 0% by mass. In some aspects, the cold solution comprises sodium methylcellulose at about 5% by mass or lower, for example at about 5, 4.5, 4, 3.5, 3, 2.5, 2, 1.5, 1, 0.5, or 0% by mass. In some aspects, the cold solution comprises a sodium alginate at about 5% by mass or lower, for example at about 5, 4.5, 4, 3.5, 3, 2.5, 2, 1.5, 1, 0.5, or 0% by mass. In some aspects, the cold solution comprises polyvinyl alcohol at about 5% by mass or lower, for example at about 5, 4.5, 4, 3.5, 3, 2.5, 2, 1.5, 1, 0.5, or 0% by mass. In some aspects, the cold solution comprises polyvinyl pyrrolidone (PVP) at about 5% by mass or lower, for example at about 5, 4.5, 4, 3.5, 3, 2.5, 2, 1.5, 1, 0.5, or 0% by mass. In some aspects, the cold solution comprises Xanthan Gum at about 0.75% by mass or lower, for example at about 0.7, 0.6, 0.5, 0.4, 0.3, 0.2, 0.1, 0.05 or 0% by mass. In some aspects, the cold solution comprises CMC at about 0.75% by mass or lower, for example at about 0.7, 0.6, 0.5, 0.4, 0.3, 0.2, 0.1, 0.05 or 0% by mass. In some aspects, the cold solution comprises guar gum at about 1% by mass or lower, for example at about 0.9, 0.8, 0.7, 0.6, 0.5, 0.4, 0.3, 0.2, 0.1, 0.05 or 0% by mass. In some aspects, the cold solution comprises locust bean gum at about 1% by mass or lower, for example at about 0.9, 0.8, 0.7, 0.6, 0.5, 0.4, 0.3, 0.2, 0.1, 0.05 or 0% by mass. In some aspects, the cold solution comprises gum tracanth at about 1% by mass or lower, for example at about 0.9, 0.8, 0.7, 0.6, 0.5, 0.4, 0.3, 0.2, 0.1, 0.05 or 0% by mass. In some aspects, the cold solution comprises carbomer at about 1% by mass or lower, for example at about 0.9, 0.8, 0.7, 0.6, 0.5, 0.4, 0.3, 0.2, 0.1, 0.05 or 0% by mass.

Additional exemplary additives include bulking agents, such as sucrose, lactose, trehalose, mannitol, sorbitol, glucose, raffinose, glycine, histidine, PVP (K40); salts such as potassium, calcium, magnesium, hydrogen phosphate, hydrogen carbonate; buffering agents, such as sodium citrate, sodium phosphate, sodium hydroxide, tris base-65, tris acetate, tris HCl-65; tonicity modifiers, such as dextrose; collapse temperature modifiers such as dextran, ficoll, gelatin, and hydroxyethyl starch; antimicrobial preservatives such as benzalkonium chloride, benzethonium chloride, benzyl alcohol, chlorobutanol, m-cresol, myristyl gamma-picolinium chloride, paraben methyl, paraben propyl, phenol, 2-phenoxyethanol, phenyl mercuric nitrate, and thimerosal; chelating agents such as calcium disodium EDTA (ethylenediaminetetra acetic acid), disodium EDTA, calcium versetamide Na, calteridol, and DTPA; antioxidant and reducing agents such as acetone sodium bisulfate, argon, ascorbyl palmitate, ascorbate (sodium/acid), bisulfite sodium, butylated hydroxyl anisole, butylated hydroxyl toluene (BHT), cystein/cysteinateHCl, dithionite sodium, gentistic acid, gentistic acid ethanolamine, glutamate monosodium, glutathione, formaldehyde sulfoxylate sodium, metabisulfite potassium, metabisulfite sodium, methionine, monothioglycerol(thioglycerol), nitrogen, propyl gallate, sulfite sodium, tocopherol alpha, alpha tocopherol hydrogen succinate, thioglycolate sodium, thiourea, and anhydrous stannous chloride; solvents and co-solvents such as benzyl benzoate, oils, castor oil, cottonseed oil, N,N dimethylacetamide, ethanol, dehydrated ethanol, glycerin/glycerol, N-methyl-2-pyrrolidone, peanut oil, PEG, PEG 300, PEG 400, PEG 600, PEG 3350, PEG 4000, poppyseed oil, propylene glycol, safflower oil, sesame oil, soybean oil, vegetable oil, oleic acid, polyoxyethylene castor, sodium acetate-anhydrous, sodium carbonate-anhydrous, triethanolamine, and deoxycholate; buffers and pH-adjusting agents such as acetate, ammonium sulfate, ammonium hydroxide, arginine, aspartic acid, benzene sulfonic acid, benzoate sodium/acid, bicarbonate-sodium, boric acid/sodium, carbonate/sodium, carbon dioxide, citrate, diethanolamine, glucono delta lactone, glycine/glycine HCl, histidine/histidine HCl, hydrochloric acid, hydrobromic acid, lysine (L), maleic acid, meglumine, methanesulfonic acid, monoethanolamine, phosphate (acid, monobasic potassium, dibasic potassium, monobasic sodium, dibasic sodium and tribasic sodium), sodium hydroxide, succinate sodium/disodium, sulfuric acid, tartarate sodium/acid, and tromethamine (Tris); stabilizers such as aminoethyl sulfonic acid, asepsis sodium bicarbonate, L-cysteine, dietholamine, diethylenetriaminepentacetic acid, ferric chloride, albumin, hydrolyzed gelatin, insitol, and D,L-methionine; surfactants such as polyoxyethylene sorbitan monooleate (TWEEN® 80), Sorbitan monooleate, polyoxyethylene sorbitan monolaurate (TWEEN® 20), lecithin, polyoxyethylene-polyoxypropylene copolymers (PLURONICS®), polyoxyethylene monolaurate, phosphatidylcholines, glyceryl fatty acid esters, urea; complexing/dispersing agents such as cyclodextrins (e.g., hydroxypropyl-B-cyclodextrin, sulfobutylether-Bcyclodextrin); viscosity building agents such as celluloses such as sodium carboxymethylcellulose (CMC), hydroxyethylcellulose, hydroxypropylmethylcellulose, methylcellulose), acacia, gelatin, methyl cellulose, xanthan gum, polyethylene glycol, guar gum, locust bean gum, carrageenan, alginic acid, gelatin, carbopol, polyvinyl and pyrrolidone. Additives can be any of those found in Sougata Pramanick et al., “Excipient Selection in Parenteral Formulation Development,” 45(3) Pharma Times 65-77 (2013), which is incorporated herein by reference in its entirety.

The cold solution can be delivered to a subject such as a human, therefore the solution can be sterile and have an osmolality and pH such that it does not harm target or non-target tissue. In some embodiments, the cold solution may have an osmolality of less than about 2,200 milli-Osmoles/kilogram. In some embodiments, the cold solution may have an osmolality of less than about 1,000 milli-Osmoles/kilogram. In some embodiments, the osmolality may be less than about 600 milli-Osmoles/kilogram. In some embodiments, the pH is between about 4.5 and about 9.

In some embodiments, the cold solution is substantially liquid such as the cold solution described in International Patent Application PCT/US2019/55605 filed on Oct. 10, 2019, which is incorporated by reference in its entirety herein. The cold solution can be cooled or supercooled to a temperature just before spontaneous nucleation occurs. Alternatively, the cold solution can be cooled or supercooled to a temperature approximate to or lower than where spontaneous nucleation occurs, then warmed such that all ice particles melt prior to delivery to a subject. One example of a cold solution is water that is supercooled. Water normally freezes at 273.15 K (0° C. or 32° F.), but it can be supercooled at standard pressure down to its crystal homogeneous nucleation at almost 224.8 K (−48.3° C./−55° F.). The supercooling process requires that water be pure and free of nucleation sites. This can be done by processes like reverse osmosis or chemical demineralization. Rapidly cooling of water at a rate on the order of 10\hat{ } K/s avoids crystal nucleation and water becomes a glass, i.e., an amorphous (non-crystalline) solid. The temperature of the cold solution can be cooled to a temperature ranging from at about 10° C. to at about −50° C. One or more additives can be selected and included in the cold solution to change the freezing point of the cold solution.

In some embodiments the cold solution is substantially solid, i.e., substantially ice, such as the cold solution (and methods for making the cold solution) described in U.S. Provisional Patent Application Ser. No. 62/953,272 filed on Dec. 24, 2019, which is incorporated by reference in its entirety herein. For example, the cold solution can comprise ice at a concentration of about 71% to about 100%, including 71, 75, 80, 85, 90, 91, 92, 93, 94, 95, 96, 97, 98, 99, 99.5, 99.9 and 100%. In some embodiments, the substantially solid solution may comprise 95 to 100% of ice in a solid state, e.g., an ice composition. In some embodiments, the substantially solid solution comprising 95% to 100% ice in a solid state is an ice needle composition, which may be generated and/or delivered via a cannula such as a needle.

In some embodiments, the cold solution is a slurry, including a liquid and solid ice particles, such as the slurry described in International Patent Applications PCT/US2019/54828 filed on Oct. 4, 2019 and PCT/US2015/047301 filed on Aug. 27, 2015, both of which are incorporated by reference in its entirety herein. Systems and methods for making a slurry are described in International Patent Application PCT/US2019/55633 filed on Oct. 10, 2019, which is incorporated by reference in its entirety herein. One or more additives can be selected to optimize flowability which is the ability of the slurry to flow through a device or within a subject. For example, flowability describes how easy it is for the slurry to move, either within the system for making the slurry, a delivery device for delivering the slurry such as a cannula, or within the body of a human subject. Flowability is dependent on several factors, including ice particle size, ice particle shape (as they relate to the configuration of the delivery device, for example, needle gauge) and viscosity.

The slurry includes ice particles, for example at a concentration of about 2% to about 70%. In some embodiments, the ice concentration is about 20% to about 50%, for example, at about 30% to about 40%, for example, about 31, 32, 33, 34, 35, 36, 37, 38, or 39%. The ice particles can be substantially rounded and uniform in shape and size. Ice crystal size can be based upon the size of the delivery device, for example, an ice particle size of about 100 μm may allow injection through a needle having an inside diameter of about 1.0 mm or smaller. In some embodiments, the ice particle size may be less than about 1 mm, or less than about 0.25 mm. In some embodiments, the temperature of the slurry can range from about −25° C. to about 10° C., for example, from about −6° C. to about 0° C.

The cold solution can be used to treat obstructive sleep apnea. An exemplary method includes delivering a cold solution to target tissue where the target tissue can be lipid rich tissue such as adipose or fat tissue. In some embodiments, the target tissue includes fat tissue in one or more of an airway, a neck, a thorax, a diaphragm, a face, or an abdomen. The airway includes anterolateral upper airway, pharyngeal fat pads (for example, fatty deposits in the laryngopharynx, nasopharynx, oropharynx, and palatopharynx), parapharyngeal fat pads (for example, fatty deposits in the retropalatal and retroglossal regions), fat located within the tongue (e.g., within the posterior tongue), and soft palate. The neck includes subcutaneous fat such as superficial subcutaneous fat and brown fat. In some embodiments, the target tissue includes subcutaneous fat located in the outer circumference of the neck where any one or more portions or all of the outer circumference of the neck can be treated. The abdomen includes subcutaneous fat such as superficial and deep subcutaneous fat, brown fat and visceral fat. The thorax includes intrathoracic fat or epicardial fat. The diaphragm includes ectopic fat. The target tissue can include one or more types of fat in one or more body areas and any combination thereof.

The cold solution of the present invention can be used to treat subcutaneous fat, visceral fat, epicardial fat, ectopic fat and airway fat via cryolipolysis, specifically, the selective targeting of non-adipocyte, lipid rich tissue, and remodeling of connective tissue, while avoiding non-specific hypertonic injury to tissue. Unlike conventional approaches, methods of the invention allow for selection of the treatment site and subsequent cryolipolysis, or cell death by freezing, of subcutaneous fat. Therefore, the invention allows for selective targeting of a particular treatment site in a subject for removal of fat tissue at the treatment site. Additionally, upon removal and reduction of fat tissue in the subcutaneous fat layer, there is a collagen response, thereby tightening of the skin in the treated area.

The cold solution can be used to treat at least a portion of subcutaneous fat around the circumference of a neck, and by decreasing the overall circumference of the neck, the external loading pressure on the airway is decreased, thus improving obstructive sleep apnea. Similarly, by treating at least a portion of the subcutaneous fat in the face, for example, one or both cheeks, the external loading pressure on the airway is decreased. By treating at least a portion of the subcutaneous fat in the abdomen, the external loading pressure on the lungs is decreased. The relationship between fat distributions and obstructive sleep apnea is described in Turnbull C D, et al. Relationships between MRI fat distributions and sleep apnea and obesity hypoventilation syndrome in very obese patients. Sleep and Breathing. 2018. 22(3): 673-81, which is incorporated by reference in its entirety herein. Additional disclosures regarding methods for treating subcutaneous fat via cryolipolisis are described in International Patent Application PCT/US2019/54834 filed on Oct. 4, 2019, which is incorporated by reference in its entirety herein.

The cold solution can be used to treat at least a portion of visceral fat in the abdomen to reduce the volume of visceral fat in order to improve lung volumes and reduce pharyngeal collapsibility, thus improving obstructive sleep apnea. Increased abdominal visceral fat can decrease lung volume and reduces traction on the pharynx which can increase pharyngeal collapsibility. The relationship between excess visceral fat and OSA is well characterized in pediatric obstructive sleep apnea as described in Canapari C A, et al. Relationship between Sleep Apnea, Fat Distribution, and Insulin Resistance in Obese Children. J Clin Sleep Med. 2011. 7(3): 268-73, which is incorporated by reference in its entirety herein. Additional disclosures regarding methods for treating visceral fat are described in U.S. Pat. No. 9,980,765 filed on Mar. 15, 2013, which is incorporated by reference in its entirety herein.

The cold solution of the present invention can be used to treat at least a portion of brown fat in one or more locations, for example in the neck, where the cold solution can cause non-shivering thermogenesis in the tissue thereby causing the body to burn calories and reduce fat tissue. Upon sensing coldness, the cold thermoreceptor sends a signal via the sympathetic pathway to the subject's hypothalamus, and the hypothalamus in turn stimulates brown fat tissue leading to non-shivering thermogenesis. Additional disclosures regarding methods for treating brown fat are described in International Patent Application PCT/US2018/20387 filed on Mar. 1, 2018, which is incorporated by reference in its entirety herein.

The cold solution can be used to treat airway fat in the airway to reduce the volume of airway fat and therefore reduce obstructions and any loading pressure in the airway.

The cold solution can be used to treat at least a portion of epicardial fat in the thorax to reduce the volume of epicardial fat and therefore reduce obstructions and any loading pressure in the thorax.

The cold solution can be used to treat at least a portion of ectopic fat in muscle tissue, for example, muscle tissue in the diaphragm, to reduce the volume of fat that may accumulate in the diaphragm thereby improving diaphragmatic function.

The type of cold solution(s) (i.e., substantially liquid, substantially solid, or slurry) can be selected based on the characteristics of the target tissue and desired outcome. For example, it could be advantageous to use a substantially solid solution in a small, discrete area of fat to achieve maximum fat cell death with a small volume of cold solution. As an example, an ice needle composition can be injected to one or more pharyngeal fat pads. In some embodiments, more than one cold solution can be used to treat one or more target tissues in the same or different body area. For example, a slurry comprising an ice content of about 35% and a substantially solid solution may be selected to treat an abdomen. In another example, a substantially liquid cold solution can be selected to treat visceral fat in the abdomen, and an ice needle composition can be used to treat one or more locations in the airway. The method of the present invention can include delivering one or more types of cold solution to the target tissue where the target tissue can include one or more types of fat in one or more body areas and any combination thereof. For example, a single treatment can include the use of one or more types of cold solution to be delivered to one or more types of types of fat in one or more body areas and any combination thereof.

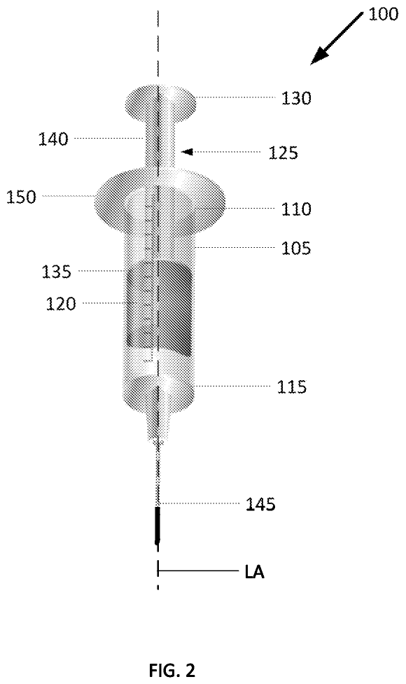

In some embodiments, delivering a cold solution can comprise injecting a cold solution via a delivery device. Any suitable delivery device may be used to deliver the cold solution to a subject. An exemplary device for delivering cold solution is generally shown in FIG. 2. The delivery device 100 includes a cylindrical member 105 having a first end 110 and a second end 115 along a longitudinal axis LA. The delivery device also includes an interior lumen 120 defined by the interior wall of the cylindrical member 105 and provided to receive and hold cold solution. The cylindrical member also includes a ledge 150, or flange, extending around the first end 110 out from the cylindrical member 105 along a plane that is orthogonal to the longitudinal axis LA. The ledge 150 also has an opening concentric with the interior lumen 120. The ledge helps facilitate handling and delivery of cold solution from the delivery device 100. In one embodiment, the delivery device 100 is a syringe-type device, for example, any suitable sterile syringe. The syringe can include a gauge size ranging from 8-25 G.

The cylindrical member 105 can be made of any type of biocompatible pharmacologically inert material suitable for use in holding and supplying fluids to be provided within a human body. Exemplary materials for the cylindrical member 105 include plastic, such as polyethylene or polypropylene, and glass. The delivery device 100 can be any size that suitable to hold one or more aliquots (doses) of cold solution for delivery to the desired tissue. As an example, the volume capacity of the delivery device 100 can be between 1 ml and 60 ml, although capacity outside of those volumes is also contemplated.

The delivery device 100 also includes a plunger 125 at least partially disposed within the interior lumen 120. The plunger 125 is configured to move in and out of the cylindrical member 105 through the first end 110. The plunger 125 includes a head 130, a plunging member 135, and a rod 140 extending between the head 130 and plunging member 135 along the longitudinal axis LA. The plunging member 135 is disposed along the rod 140 at a predetermined distance from the head 130. The delivery device 100 also includes at least one needle 145 extending from the second end 115. The needle 145 can comprise a gauge between 7 gauge and 34 gauge and a length between ¼ inch and 10 inches, such as about ¼ inch, ½ inch, 1 inch, 2 inches, 3 inches, 4 inches, 5 inches, 6 inches, 7 inches, 8 inches, 9 inches, or 10 inches. In some embodiments, the cylindrical member 105 narrows or tapers to a small opening at the second end 115, where the small opening is configured to receive the needle 145. Preferably, the needle 145 is a hypodermic needle. Exemplary needle materials include, but are not limited to, stainless steel and carbon steel, with or without nickel plating.

The plunger 125, including the head 130 and the rod 140, can be any type of biocompatible, pharmacologically inert material suitable for coming in contact with fluids to be provided within a human body. Exemplary materials for the plunger 125 include plastic, such as polyethylene or polypropylene, and glass. With respect to the plunging member, a portion or all of the plunging member 135 can be a rubber material, such that a seal is formed between the sides of the plunging member 135 and the interior wall of the cylindrical member 105. The rubber material can be any rubber suitable for coming in contact with fluids to be provided to the human body, such as natural rubber latex or a synthetic rubber. In some embodiments, the delivery device 100 can also include an agitator (not shown) disposed within the interior lumen 120 configured to mix the cold solution ingredients.

Once the cold solution is ready for delivery to tissue using the delivery device 100, the needle 145 is used to pierce the skin. Once the needle 145 is through the skin and positioned at or near the target tissue, the plunger 125 is forced downward toward the second end 115 of the cylindrical member 105. The force of the plunging member 135 on the cold solution forces the cold solution through the cylindrical member 105, out the needle 145, and into (or near) the target tissue. In one embodiment, more than one needle is provided at the second end 115 of the delivery device 100. The more than one needle can be provided in single row array, multiple row array, circular pattern, or any other conceivable arrangement.

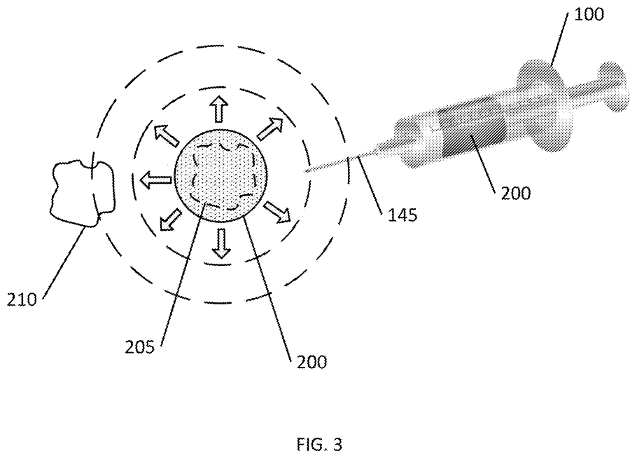

Referring to the procedure of FIG. 3, the device 100 of FIG. 2 is used to deliver cold solution 200 to fat tissue 205. In other examples, cold solution can be delivered using a syringe-type device, a catheter or a cannula. The needle 145 is inserted through the subject's skin and advanced to a location at or near the target fat tissue 205 (shown in phantom line). The cold solution 200 is then delivered and cools the fat tissue 205.

After delivery, an area affected by the cold solution 200 expands to a size larger than the initial delivery site (shown in the figure as arrows radiating outwardly from the delivered cold solution 200 and dashed circles of increasing size). The cooling effect of the cold solution 200 is localized to the fat tissue 205 and possibly surrounding tissue, such as adjacent tissue 210. In this way, discomfort caused by the cold treatment is limited. The cold solution 200 is sterile and biocompatible; and, as such, the cold solution 200 can be left in the body (e.g. no removal of the cold solution is necessary after cooling has been effected). In some embodiments, the cold solution can be removed from the body after the cooling has been effected. For example, after delivery to the airway and cooling has been effected, it may be desirable to remove at least a portion of the cold solution. The cold solution can be removed by any suitable means, for example via absorbing, e.g., via a sponge-like material, or withdrawing, e.g., using a syringe or suction.

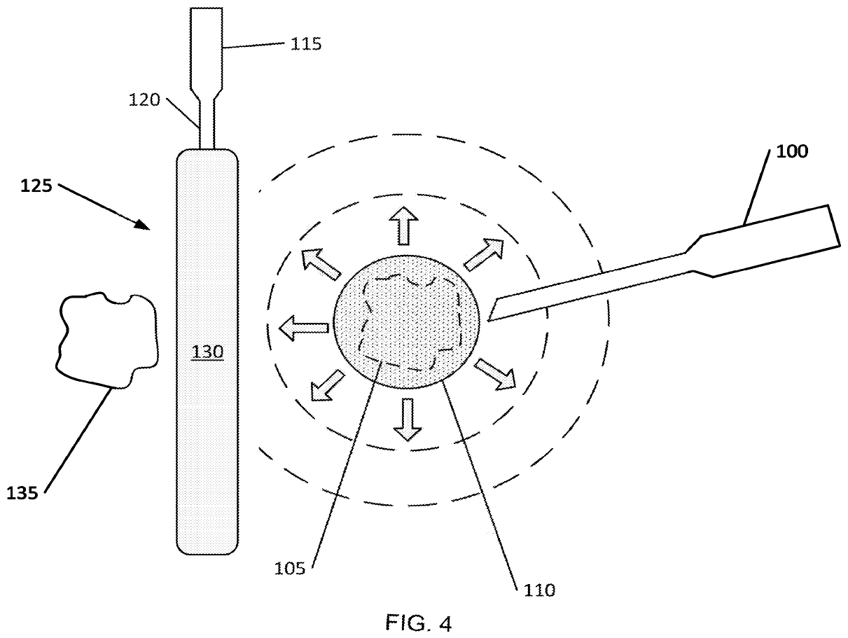

In some embodiments, a cold solution containment device can be used in combination with the delivery device 100, for example, a device comprising a balloon configured for controlling the cooling effect of the cold solution, as shown in FIG. 4. As an example, a balloon deployment device 115 having an application cannula 120 is inserted through the patient's skin. At the distal end of the application cannula 120, there is a controlling end 125. The deployment device 115 is advanced until the controlling end 125 is at a location between the target tissue 105 and an adjacent (surrounding) tissue 135. The controlling end 125 includes a balloon 130. While the balloon 130 is shown having a linear shape, it can have any shape, such as a ring that encircles the target tissue 105. In some embodiments, the balloon 130 is filled with air to create a barrier between the adjacent tissue 135 and the spreading cold solution 110. The balloon 130 limits heat transferring from the adjacent tissue 135 to the cold solution 110. In some embodiments, the delivery device 100 comprises a cannula such as a needle. In some embodiments, the balloon deployment device is the delivery device, for example, balloon 130 can be filled with cold solution so as to deliver and contain the solution to a particular area. In some embodiments, the balloon can be punctured to release the cold solution to directly contact the target tissue. The puncturing can be by any suitable means, for example via a needle passed through the cannula.

Any suitable volume of cold solution can be delivered based on the characteristics of the target tissue and desired effect. A treatment may include delivering a volume of cold solution to one or more treatment sites within a target tissue area. For example, when a cold solution is delivered via injection, the target tissue may be treated via one or more injection sites, i.e., puncture site, and one or more deposition sites. The deposition site is where the cold solution is deposited, regardless of the injection site, and may be a different site than the injection site or the same site. One or more treatments can be required to achieve a desired effect.

For example, where the target tissue is fat tissue such as subcutaneous fat, visceral fat or brown fat in the abdomen and the delivery comprises injection, the amount of cold solution injected may be about 2 L or less per injection site. In some examples, the amount of cold solution injected is about 1 mL to about 2 L per injection site. For example, different patients have different amounts of subcutaneous fat, and therefore, some patients may require injection of greater amounts of cold solution in order to produce visible effects of reduction and removal of subcutaneous fat. Other patients may require multiple treatments to produce effects of removal or reduction of subcutaneous fat or tightening of the skin as a result of a collagen response. A cold solution may also be injected at one or more treatment sites. For example, the selected treatment sites may be the superficial subcutaneous fat layer, the deep subcutaneous fat layer, or both. For example, the cold solution may be injected into the superficial subcutaneous fat layer or deep subcutaneous fat layer at a plurality of injection sites. In some embodiments, the cold solution is injected at a plurality of injection sites into both subcutaneous fat layers.

As another example, where the target tissue is fat tissue in the neck, such as subcutaneous fat or brown fat, the amount of cold solution injected may be about 1 mL to about 1 L per injection site. For example, a cold solution can be delivered to the entire outer circumference of the neck or any portion or portions thereof.

As compared to the abdomen and neck, a smaller volume of cold solution may be delivered to the airway, for example, 1 mL may be delivered per injection site. For example, a cold solution can be delivered to one or more locations in the airway, such as any one of the anterolateral upper airway, pharyngeal fat pads (for example, fatty deposits in the laryngopharynx, nasopharynx, oropharynx, and palatopharynx), parapharyngeal fat pads (for example, fatty deposits in the retropalatal and retroglossal regions), fat located within the tongue (e.g., within the posterior tongue), and soft palate. Injections can be made in the mouth or through the neck. Access to a treatment site through the mouth can be facilitated by a scope.

In an example, the injection sites may form a pattern, such as a plow, fan, or grid-like pattern, or in a single bolus or multiple bolus injections. In another example, one injection site is used repeatedly, thereby reducing the number of injection sites and concomitant scarring potential. In a plow injection pattern, a single initial target injection site is used followed by a moving needle for additional deposition sites, for example in a linear pattern. In a fan injection pattern, deposition sites form an arc from 1 to 360 degrees. In a bolus injection, the cold solution is deposited in a single injection site.

The injection pattern and/or cold solution (including type and/or ingredients) can be determined based on the subject's profile, treatment plan (as described below), or based on the target site to be treated. For example, an injection pattern and/or volume may be selected to optimize consistency of temperature at the target site. In an embodiment, the injection pattern and/or volume is selected in order to achieve gradient cooling of fat proximate to a target site or injection site. Injection techniques, including the patterns described herein, are known to those of skill in the art.

In some embodiments, delivering a cold solution to a target tissue can include topically contacting the cold solution to the target tissue. For example, the delivering of the cold solution can include contacting the target tissue and circulating the cold solution through a delivery device, for example, the delivery device described in FIG. 4 having a balloon. Alternatively, the cold solution can be circulated through a tubular member such as a catheter. This may be desirable for target tissue that is more difficult to reach, for example, in the lower airway or thorax. In another example, the delivering of the cold solution can include contacting the target tissue and directly applying cold to the target tissue, for example, via a patch comprising the cold solution. The patch can include an adhesive configured to adhere to the target tissue.

In some embodiments, delivering a cold solution to a target tissue can include creating an incision and positioning the cold solution at or near the target tissue. For example, when the cold solution is a substantially solid composition, an incision can be made in the target tissue area, such as an abdomen, and the substantially solid composition can be positioned at or near the target tissue.

Optionally, the method may include one or more of pre-, peri- and post-treatment monitoring such as imaging and/or performing one or more measurements. In some aspects, the method can include determining the subject's weight or BMI before and/or after treatment and comparing the subject's weight or BMI before treatment to the weight or BMI after treatment. For example, an indication of success would be an observation of a decrease in weight or BMI, decrease in measurements of girth, e.g., waist, chest, hip, thigh, or arm circumference and/or improvement in snoring and/or a performance of a sleep study.

The method can include assessing the amount fat tissue in the subject and recording these observations. The evaluation can be performed before, during, and/or after the delivery of cold solution. For example, the evaluation can be performed at least 1 day, 2 days, 4, 7, 14, 21, 30, 60, 90, 120 or more days before and/or after the delivery of cold solution. In some instances, the subject will have decreased amount of fat tissue. In other instances, the subject will show reduced symptoms.

This information can be used to determine the future course of treatment for the subject. For example, treatment may be continued without change, continued with change (e.g., additional treatment or more aggressive treatment such as an increase in volume delivered or a cold solution comprising different ingredients), or treatment can be stopped.

Optionally, a treatment plan can be created for a subject, for example to determine one or more of type of cold solution, cold solution properties (for example, ingredients, tonicity and/or ice content), target tissue site(s) (e.g., type of fat in a body location such as subcutaneous fat in the abdomen), volume of cold solution to be delivered to each site, and delivery method(s) (e.g., injection, topical/contact, and/or incision). A single treatment can include delivering cold solution to one or more treatment or target tissue sites. One or more cold solution types and corresponding volumes and delivery methods can be used for a single treatment site or multiple treatments sites in a single treatment. Factors considered in creating a treatment plan for a subject may comprise one or more of gender, height, body weight, body fat percentage, anatomy, lifestyle, vitals, medical history, lipid profiles, skin elasticity, medication, nutrition, supplements, demographic, fat saturation, sleep study observations, and the like. Fat saturation may be characterized by one or more of imaging, biopsy, and impedance measurement. As an example, once a plan is created for the subject, the amount and type of cold solution to be administered can be adjusted based on one or more of the area or areas to be treated, the depth of injection, and the injection pattern to be used.

A computer or artificial intelligence system may be utilized to create a treatment plan for a subject by collecting pre-, peri-, and/or post-injection data from multiple subjects. It is appreciated that the more data points, the more effective the artificial intelligence system will be in creating a treatment plan for a subject. For example, pre-, peri-, and/or post-injection data may be collected for each subject comprising one or more of gender, height, body weight, body fat percentage, the subject's anatomy, lifestyle, the subject's vitals, medical history, lipid profiles, skin elasticity, medication, nutrition, supplements, demographic, fat saturation, imaging data, treatment data and fat loss data. Data may be measured by any suitable means. For example, fat loss data may be measured by calipers, cutometers, and/or any imaging method such as ultrasound, MM, 3D photography, visual assessment, and the like.

An exemplary treatment method can include the following steps: assessing a subject; identifying the target tissue(s); creating a treatment plan; preparing the cold solution; cleaning the area of the subject's skin overlying the target tissue; marking an entry point on the skin through which a delivery device for delivering cold solution will enter (the entry point can be identified visually, or through the use of one or more imaging technique such as ultrasound, magnetic resonance and x-ray); inserting the delivery device into the entry point and advancing to the target tissue; and injecting the cold solution at (or near) the target tissue. Any suitable amount of cold solution can be delivered to multiple sites at (or near) the target tissue, and in some instances, injection to multiple sites increases the amount of target tissue that is exposed to the cold solution and cooled and can improve the effectiveness of the treatment. The cold solution can be delivered using one or more injection patterns, for example, one or more boluses, a plow, fan, or grid-like pattern, or other injection techniques known to those of skill in the art. Optionally, a massaging step post injection may be utilized to increase fat cell damage. Additionally, post-treatment monitoring and follow up can be performed; and optionally additionally treatments may be performed for the same or different target tissue.

In some aspects, the method can include performing a 3D face scan to screen or determine if a subject has sleep apnea, to identify the target tissue(s), and/or assess the outcome of a treatment. An exemplary 3D scanning tool can measure linear distances and angles, and geodesic distances between anatomical landmarks from 3D photographs, and is described in Eastwood P, Gilani S Z, McArdle N, et al. Predicting sleep apnea from three-dimensional face photography. J Clin Sleep Med. 2020; 16(4):493-502, which is incorporated by reference in its entirety herein.

As discussed above, the target tissue can include at least a portion in any one of airway fat, subcutaneous fat, visceral fat, brown fat, epicardial fat, and/or ectopic fat in one or more locations in the body. For example, after an assessment, it may be determined that a treatment includes delivering a cold solution to subcutaneous fat in the entire circumference of the neck, subcutaneous fat in the abdomen, and airway fat in the soft palate. For a different subject, the treatment may be different, for example, a treatment may include delivering a cold solution to visceral fat in the abdomen only. The method of the present invention can include delivering a cold solution to target tissue where target tissue can include one or more types of fat in one or more body areas and any combination thereof.

Delivering a cold solution to a target tissue to treat obstructive sleep apnea can be accompanied by one or more additional treatments, for example, a muscle toning treatment to the airway, neck, and/or abdomen. By toning the muscle in the area, the fat tissue may be further reduced, and the skin tightened, thereby improving the clinical outcome. Exemplary muscle toning treatments to the airway include tongue and/or jaw exercises. Exemplary muscle toning treatments to the abdomen include any known abdominal exercise such as sit ups and/or use of an abdominal exercise device utilizing vibration and/or electrical stimulation. The methods disclosed herein may also be used in combination with one or more known obstructive sleep apnea devices such as a positive airway pressure device, a mouthguard or an airway stimulator device.

Treating obstructive sleep apnea may also play a role in treating one or more of diabetes, atrial fibrillation, stroke, and nonalcoholic steatohepatitis (or nonalcoholic fatty liver disease).

In addition to treating obstructive airway disease such as obstructive sleep apnea, additional obstructive airway diseases linked to obesity, such as asthma, can be treated using the disclosed cold solutions and methods. For example, a study demonstrated that fat tissue within the airway wall in individuals with elevated BMI which correlates to greater wall thickness and airway inflammation. See Elliot J G, Donovan G M, Wang K C W, et al. Fatty Airways: Implications for Obstructive Disease. Eur Respir J 2019; in press (https://doi.org/10.1183/13993003.00857-2019) which is incorporated by reference in its entirety herein.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method of treating obstructive sleep apnea comprising delivering a cold solution to target tissue wherein the target tissue comprises fat tissue in one or more of an airway, a neck, a thorax, a diaphragm, a face, or an abdomen.

2. The method of claim 1, further comprising cooling the target tissue via the cold solution.

3. The method of claim 1, further comprising performing at least one of pre-, peri- or post-procedure imaging and/or measurements.

4. The method of claim 1, further comprising creating a treatment plan.

5. The method of claim 1, wherein the delivering comprises delivering the cold solution to one or more sites in the target tissue.

6. The method of claim 1, further comprising removing at least a portion of the cold solution from the target tissue.

7. The method of claim 6, wherein the removing comprises withdrawing the cold solution via a syringe.

8. The method of claim 6, wherein the removing comprises withdrawing the cold solution via suction.

9. The method of claim 6, wherein the removing comprises absorbing the cold solution.

10. The method of claim 1, wherein the delivering a cold solution comprises injecting the cold solution to one or more sites in the target tissue.

11. The method of claim 10, wherein the injecting is performed via a delivery device.

12. The method of claim 11, wherein the delivery device comprises a cannula.

13. The method of claim 12, wherein the delivery device comprises a syringe having a single needle.

14. The method of claim 13, wherein the syringe comprises an array of needles.

15. The method of claim 14, wherein the array of needles is expandable.

16. The method of claim 11, wherein the delivery device comprises a balloon.

17. The method of claim 16, wherein the balloon is configured to protect non-target tissue from exposure to the cold solution.

18. The method of claim 16, wherein the balloon is configured to contain the cold solution prior to delivery.

19. The method of claim 10, wherein the injecting comprises injecting one or more boluses of cold solution.

20. The method of claim 10, wherein the injecting comprises injecting a cold solution in a pattern.

21. The method of claim 1, wherein the delivering a cold solution comprises circulating a cold solution proximate to the target tissue.

22. The method of claim 1, wherein the delivering a cold solution comprises topically contacting the cold solution to the target tissue.

23. The method of claim 1, wherein the airway comprises an anterolateral upper airway; pharyngeal fat pads; parapharyngeal fat pads; fat located within the tongue; and a soft palate, and the delivering a cold solution to target tissue comprises delivering a cold solution to one or more portions of the airway.

24. The method of claim 1, wherein the neck comprises subcutaneous fat in an outer circumference of the neck, and the delivering a cold solution to target tissue comprises delivering a cold solution to subcutaneous fat in at least a portion of the outer circumference of the neck.

25. The method of claim 1, wherein the neck comprises subcutaneous fat in an outer circumference of the neck, and the delivering a cold solution to target tissue comprises delivering a cold solution to subcutaneous fat in one or more portions of the outer circumference of the neck.

26. The method of claim 1, wherein the neck comprises subcutaneous fat in an outer circumference of the neck, and the delivering a cold solution to target tissue comprises delivering a cold solution to subcutaneous fat in the entire outer circumference of the neck.

27. The method of claim 1, wherein the neck comprises brown fat, and the delivering a cold solution to target tissue comprises delivering a cold solution to at least a portion of the brown fat.

28. The method of claim 1, wherein the abdomen comprises subcutaneous fat, and the delivering a cold solution to target tissue comprises delivering a cold solution to at least a portion of the subcutaneous fat.

29. The method of claim 1, wherein the abdomen comprises brown fat, and the delivering a cold solution to target tissue comprises delivering a cold solution to at least a portion of the brown fat.

30. The method of claim 1, wherein the abdomen comprises visceral fat, and the delivering a cold solution to target tissue comprises delivering a cold solution to at least a portion of the visceral fat.

31. The method of claim 1, wherein the thorax comprises epicardial fat, and the delivering a cold solution to target tissue comprises delivering a cold solution to at least a portion of the epicardial fat.

32. The method of claim 1, wherein the diaphragm comprises ectopic fat, and the delivering a cold solution to target tissue comprises delivering a cold solution to at least a portion of the ectopic fat.

33. The method of claim 1, wherein the face comprises subcutaneous fat, and the delivering a cold solution to target tissue comprises delivering a cold solution to at least a portion of the subcutaneous fat.

34. The method of claim 1, further comprising toning a muscle in the target tissue.

35. The method of claim 1, further comprising providing a positive airway pressure device.

36. The method of claim 1, further comprising providing a mouthguard.

37. The method of claim 1, further comprising providing an airway stimulator.

38. The method of claim 1, wherein the method further treats one or more of asthma, diabetes, atrial fibrillation, stroke, and nonalcoholic steatohepatitis.

39. A method of treating obstructive sleep apnea comprising delivering a cold solution to target tissue wherein the target tissue comprises one or more of airway fat, subcutaneous fat, visceral fat, brown fat, epicardial fat, or ectopic fat.

40. A cold solution for treating obstructive sleep apnea comprising water and optionally one or more additives.

41. The cold solution of claim 40, wherein the cold solution comprises one or more of a salt, a sugar or a thickener.

42. The cold solution of claim 40, wherein the cold solution comprises an osmolality of less than about 2,200 milli-Osmoles/kilogram.

43. The cold solution of claim 40, wherein the cold solution comprises an osmolality of less than about 1,000 milli-Osmoles/kilogram.

44. The cold solution of claim 40, wherein the cold solution comprises an osmolality of less than about 600 milli-Osmoles/kilogram.

45. The cold solution of claim 40, wherein the cold solution comprises a pH of about 4.5 to about 9.

46. The cold solution of claim 40, wherein the cold solution comprises no ice.

47. The cold solution of claim 40, wherein the cold solution comprises ice particles at a concentration of about 2% to about 70%.

48. The cold solution of claim 40, wherein the cold solution comprises ice particles at a concentration of about 20% to about 50%.

49. The cold solution of claim 40, wherein the cold solution comprises ice particles at a concentration of about 71% to about 100%.

50. The cold solution of claim 40, wherein the cold solution comprises ice particles at a concentration of at least about 95%.

51. The cold solution of claim 40, wherein the cold solution comprises substantially ice.
